(19) United States
(12) Patent Application Publication
(10) Pub. No.: US 2021/0023155 A1
(43) Pub. Date: Jan. 28, 2021

(54) CANNABIS SCREW CONVEYOR PROCESSOR AND MEMBRANE SYSTEM AND METHODS

(71) Applicant: Marquette Analytica, Midland, MI (US)

(72) Inventor: Robert Opperman, Midland, MI (US)

(73) Assignee: Marquette Analytica, Midland, MI (US)

(21) Appl. No.: 16/935,589

(22) Filed: Jul. 22, 2020

Related U.S. Application Data
(60) Provisional application No. 62/877,079, filed on Jul. 22, 2019.
(60) Provisional application No. 62/903,497, filed on Sep. 20, 2019.
(60) Provisional application No. 62/936,572, filed on Nov. 17, 2019.

(51) Int. Cl. A61K 36/185; A61K 31/05

(52) U.S. Cl. CPC A61K 36/185; A61K 2236/33; A61K 2236/15; A61K 31/05

(57) ABSTRACT

The disclosure relates to a continuous Cannabis plant material processing system comprising ultrasonic treatment of Cannabis plant material coupled to a membrane system for the efficient recovery of cannabinoids.

### CROSS REFERENCE TO RELATED PATENT APPLICATIONS

This patent application claims priority to U.S. Provisional Patent Application No. 62/877,079, filed Jul. 22, 2019; U.S. Provisional Patent Application No. 62/903,497, filed Sep. 20, 2019; U.S. Provisional Patent Application No. 62/936,572, filed Nov. 17, 2019, the disclosures of each of which are herein incorporated by reference in their entirety.

### BACKGROUND

Cannabis has been used as a source of fiber to make paper and clothing, as a recreational drug, and in traditional medicine. In recent years, compounds present in Cannabis, including the cannabinoids Δ⁹-tetrahydrocannabinol (THC) and cannabidiol (CBD), have been shown to alleviate inflammation and cancer-related symptoms. See, e.g., Federica Pellati et al., Cannabis sativa L. and Nonpsychoactive Cannabinoids: Their Chemistry and Role against Oxidative Stress, Inflammation, and Cancer, BIOMED RES. INT'L, 2018.

Historically, Cannabis has been divided into three species: Cannabis sativa L., Cannabis indica Lam., and Cannabis ruderalis Janisch. These species have been extensively hybridized, and the resulting hybrids are classified as C. sativa and can be further characterized by chemotype according to the cannabinoid profile.

Cannabis plants having high THC levels are often studied for medicinal properties. In contrast, Cannabis plants having low THC levels (hemp) and high CBD levels have been used in textiles and foods. However, interest in CBD has grown, because it is non-psychoactive and elicits anti-inflammatory, anxiolytic, and anticonvulsant properties. For example, the United States Food and Drug Administration has approved synthetic cannabidiol (EPIDIOLEX®) for the treatment of seizures associated with Lennox-Gastaut syndrome or Dravet syndrome in children.

The increased interest in CBD as well as the lift of the ban on industrial hemp (Cannabis having a THC content of less than 0.3%) in the United States and Europe has made industrial hemp an attractive source of fiber, cannabinoids, and other phytochemicals. In addition to cannabinoids, Cannabis plants contain cannabinoid acids, terpenes, flavonoids, carbohydrates, fatty acids, and phenolic compounds. Cannabis-derived terpenes impart Cannabis's distinctive smell and have been shown to reduce cytokines associated with peripheral inflammation. Furthermore, research into derivatives of compounds from Cannabis plants require large amounts of each starting material. However, there have been challenges in extracting these compounds in high yields and purity.

The current methodology used for the extraction of cannabinoids from Cannabis (including hemp) require the transportation of the harvested biomass to an extraction facility, which then deploys various means of extraction such as hydrocarbon and liquid carbon dioxide solvents. For these processes to function efficiently, the biomass must be dried (dehydrated) to <10% moisture and, prior to extraction, the biomass materials must be preserved to minimize the degradation and/or sublimation of the cannabinoid materials as it awaits extraction. These preparations are costly and are not conducive to scaling to a commercially successful industrial sized operation.

There exists a need in the art for more efficient methods of rapid preparation of cannabinoid extracts from Cannabis plant material with high yields, substantially free from contaminants.

### SUMMARY

The disclosure provides, among other things, methods of preparing cannabinoid extracts that advantageously increase cannabinoid yields and decrease process time from hours to minutes. The efficiency of using membrane separation processes in combination with ultrasonic cavitation (e.g., using an aqueous medium) is greater than current methods that require longer time periods, the use of organic solvents, and higher costs. For example, the system and methods disclosed herein scale more easily than current methods and can be used in both batch and continuous processes. The solvent recovery methods of the disclosure are also safer than distillation, have fewer moving parts, and reduce refrigeration and heating demands. Additionally, the system and methods described herein advantageously increases yields from hemp without the need to increase cannabinoid production by the hemp plants using genetic techniques. For example, the high efficiency of cannabinoid extraction increases the economic value of processing low-cannabinoid Cannabis, e.g., male plants, fertilized female plants, hemp aged over 6 months post-harvest.

The disclosure describes a discovery that high yields of cannabinoid extracts can be obtained using the system and methods described herein without pre-treating a crude extract with an adsorbent. Surprisingly, it has been discovered that pesticides/fungicides were retained by the dewaxing membrane with the lipids without the need for a separate adsorption step. Also surprising was that fouling of the membranes remains low and overall efficiency remains high without pre-treating the miscella stream. This miscella stream may then be subjected to decarboxylation by heat and crystallization to yield high purity cannabinoids, e.g., cannabidiol (CBD).

In one embodiment, the invention provides a method for processing Cannabis plant material comprising: applying ultrasonic cavitation with an ultrasonic cavitation device to a mixture comprising Cannabis plant material and at least one solvent in an extraction tank, to produce a miscella stream; the extraction tank having a first end proximal to the ultrasonic cavitation device and a second end distal to the ultrasonic cavitation device; and filtering the miscella stream through at least one membrane to produce a cannabinoid extract. In a further embodiment, the screw conveyor has a central axis, the central axis making an incline angle θ of about 1° to about 90° C. relative to a horizontal plane intersecting the central axis. In another embodiment, the incline angle is between 80° and 90°. The extraction tank may comprise a screw conveyor located inside the extraction tank; the screw conveyor capable of moving the Cannabis plant material from the first end to the second end. The screw conveyor may be a varied geometry screw conveyor. In an embodiment, the extraction tank may comprise a centrifuge.

In one embodiment, the invention provides a method for processing Cannabis plant material comprising: applying ultrasonic cavitation with an ultrasonic cavitation device to a mixture comprising Cannabis plant material and at least one solvent in an extraction tank, to produce a miscella stream; the extraction tank comprising a centrifuge; and filtering the miscella stream through at least one membrane to produce a cannabinoid extract. In an embodiment, the miscella stream may be filtered through one, two, or three membranes to produce a cannabinoid extract.

In an embodiment, a Cannabis processing system may comprise an extraction tank configured with a varied geometry screw conveyor arranged at an angle is coupled to a biomass input at the proximal end and a biomass output at the distal end, wherein the extraction tank is arranged on an incline of 1° to 90° C., with the lowest end at the proximal end, coupled to an ultrasonic horn situated in close proximity, preferably 0.1-10 cm, e.g., 1-5 cm, from a miscella drain, wherein the solution level is highest at the proximal end with the ultrasonic horn and miscella drain and lowest at the distal end with the biomass output, wherein the varied geometry screw conveyor has steadily decreasing pitch between the blades as to configured to compress the biomass as it passes from the biomass input at the proximal end to the biomass output at the distal end, a miscella drain coupled to a miscella stream output coupled to a membrane pump that pressurizes the miscella stream and sends it through a double membrane system, wherein the first membrane is a dewaxing membrane that removes lipids from the miscella stream as the first retentate and a desolvent membrane that removes the cannabinoids from the miscella stream as the second retentate, wherein the second permeate comprising the aqueous solution can be collected by a solvent reserve tank with thermostat control coupled to the membrane system, wherein the solvent reserve tank with thermostat control is configured to collect the solvent from the desovlent membrane, store it, adjust the aqueous solution temperature as necessary, and is coupled to a solvent return pump coupled to a solvent return line configured to send the recovered solvent into the extraction tank. In an embodiment, the miscella stream may be passed through a filter before being subjected to membrane filtration to obtain a cannabinoid extract. In an embodiment, at least a portion of the solvent is returned to the extraction tank before being subjected to membrane filtration. In an embodiment, at least a portion of the second permeate is returned to the first membrane to be subjected to membrane filtration for at least a second time.

### BRIEF DESCRIPTION OF THE DRAWINGS

FIG. 1A depicts an exemplary system for extracting cannabinoids from Cannabis plant material according to the disclosure. This system may be deployed as a mobile system, preferably in a C1D1 container laboratory or C1D2 container laboratory.

FIG. 1B depicts an exemplary a system for extracting cannabinoids from Cannabis plant material according to the disclosure. This system may be deployed as a mobile system, preferably in a C1D1 container laboratory or C1D2 container laboratory.

FIG. 1C depicts an exemplary extraction tank (top view), where the miscella drain is positioned between two sonication devices in the extraction tank.

FIG. 1D depicts an exemplary extraction tank (side view), where the miscella drain is coupled to on an extension, situated below the sonication device(s) in the extraction tank. The extension, optionally a pipe, may be capable of being moved to different positions within the extraction tank.

FIG. 2A is a flow-chart of the method steps for membrane filtration of a miscella stream to collect cannabinoids according to the disclosure.

FIG. 2B is a flow-chart of the method steps for membrane filtration of a miscella stream to collect cannabinoids according to the disclosure.

FIG. 3 is a flow-chart of the method steps for membrane filtration of a miscella stream to collect cannabinoids according to the disclosure.

FIG. 4 is a flow-chart of the method steps for membrane filtration of a miscella stream to collect cannabinoids according to the disclosure.

FIG. 5 depicts the extraction over time using ultrasonic cavitation or no ultrasonic cavitation.

### DETAILED DESCRIPTION

### Medical Cannabis and Cannabinoids

Medical Cannabis has been used to alleviate the symptoms of patients suffering from a variety of medical conditions including cancer, anorexia, AIDS, chronic pain, spasticity, glaucoma, arthritis, and migraines. For example, the antiemetic properties of Cannabis have been useful in the treatment of nausea and vomiting in cancer patients undergoing chemotherapy as well as in the treatment of weight loss syndrome associated with AIDS. Glaucoma patients have been treated with Cannabis to reduce intraocular pressure. Muscle relaxing and anticonvulsant effects of Cannabis have also been reported.

However, consumption of the whole Cannabis plant, e.g., by smoking, has side-effects including impaired cognitive functions, perception, reaction time, learning, and memory. To mitigate such side-effects, there is growing interest in investigating the medicinal properties of individual Cannabis-derived compounds and sub-combinations and derivatives thereof. In addition, many potential patients have personal or religious objections to consuming Cannabis in plant form. To address these issues, there is a great interest in developing a pharmaceutical form of Cannabis extracts. The disclosure relates to methods of preparing Cannabis extracts that can be useful in such applications.

Cannabinoids are synthesized primarily in the glandular trichomes of Cannabis plants and include tetrahydrocannabinolic acid, Δ⁸ tetrahydrocannabinolic acid, Δ⁹ tetrahydrocannabinolic acid, tetrahydrocannabinol, Δ⁸ tetrahydrocannabinol, Δ⁹ tetrahydrocannabinol (THC), cannabidiolic acid, cannabidiol (CBD), cannabinol, cannabigerolic acid, cannabigerol, cannabigerolic acid, cannabichromene, and tetrahydrocannabivarin, any of which can be obtained by the methods described herein.

In addition to cannabinoids, terpenes obtained by the disclosed methods may include trans-nerolidol, terpinolene, terpineol, saniene hydrate, L-fenchone, guaiol, geraniol, γ-terpinene, β-pinene, α-terpinene, α-humulene, α-cedrene, α-Pinene, R-Limonene, S-Camphor, fenchyl alcohol, R-camphor, (−)-borneol, (+)-borneol, (+)-pulegone, valencene, transcaryophyllene, p-mentha 1,5-diene, ocimene, nerol, linalool, isoborneol, hexahydrothymol, geranyl acetate, farnesene, eucalyptol, cis-nerolidol, camphor, camphene, β-myrcene, carene, (−)-isopulegol, caryophyllene oxide, α-bisabolol, (+)-fenchone, (+)-cedrol, or combinations thereof. The terpenes isolated using the methods and systems disclosed herein may be collected.

Cannabis flavonoids that can be obtained by the methods of this disclosure include quercetin, luteolin, kaempferol, cannaflavin A, apigenin, or combinations thereof. The flavonoids isolated using the methods and systems disclosed herein may be collected.

### Cannabinoid Extraction

The disclosure generally relates to improved methods for Cannabis plant material processing and cannabinoid extraction. For example, the disclosure relates to a method for processing Cannabis plant material comprising: applying ultrasonic cavitation with an ultrasonic cavitation device to a mixture comprising Cannabis plant material and at least one solvent in an extraction tank, to produce a miscella stream; the extraction tank having a first end proximal to the ultrasonic cavitation device and a second end distal to the ultrasonic cavitation device; the extraction tank comprising a screw conveyor located inside the extraction tank, the screw conveyor capable of moving the Cannabis plant material from the first end to the second end; and filtering the miscella stream through at least one membrane to produce a cannabinoid extract. The cannabinoid extract may be collected in light-proof vessels, optionally flushed with nitrogen.

In another example, the disclosure relates to a method for processing Cannabis plant material comprising: supplying Cannabis plant material to a screw conveyor arranged in a chamber comprising a sonication device configured to provide ultrasonic cavitation and admixing with an alcohol at a temperature between −20° C. and 20° C.; applying ultrasonic cavitation at a sufficient power to disrupt the trichomes and produce a miscella stream; collecting the miscella stream; moving the Cannabis plant material along the screw conveyor away from the sonication probe towards an output port; and subjecting the miscella stream to membrane filtration to produce a cannabinoid extract.

This cannabinoid extract may be heat treated to decarboxylate the cannabinoids, e.g., cannabidiol acid (CBDA) to produce cannabidiol (CBD), or tetrahydrocannabinol acid (THCA) to produce tetrahydrocannabinol (THC). The cannabinoid crystals produced by decarboxylation and crystallization can be collected, washed, tested, weighed, and packaged, preferably in nitrogen-flushed, light proof containers (e.g., vials or bags).

The cannabinoid crystals produced by decarboxylation and crystallization (e.g., CBD crystals) are collected by filtration and the solvent(s) may be subjected to membrane exchange to separate the solvent(s). In another embodiment, the cannabinoid crystals may be washed with a solvent and dried. The cannabinoid crystals collected may be washed, tested, weighed, and packaged, preferably in nitrogen-flushed, light proof containers (e.g., vials, jars, bags).

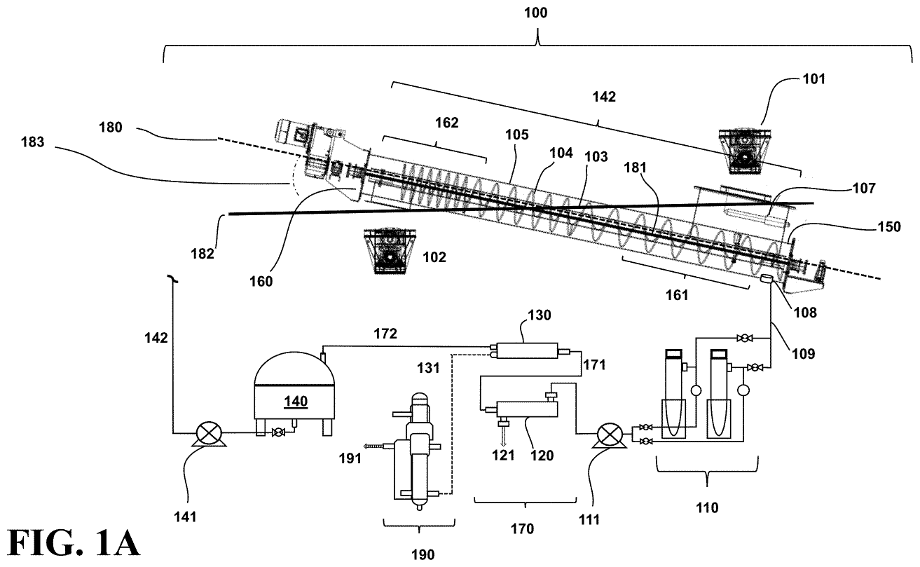

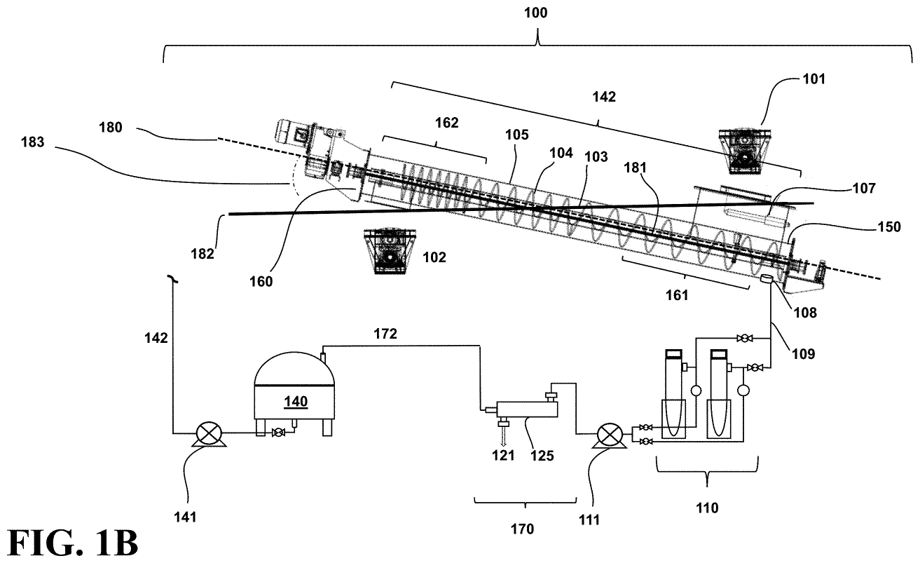

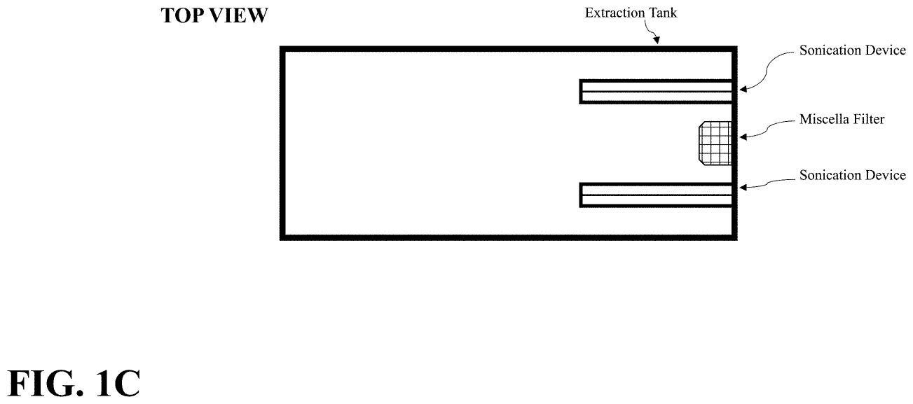

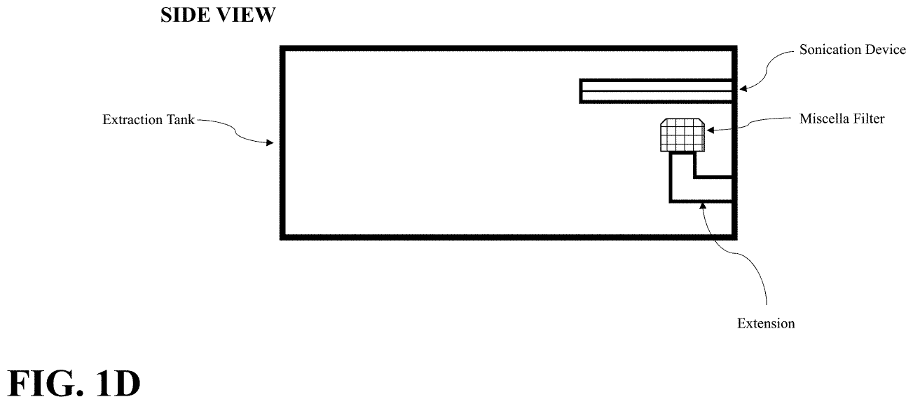

Making reference to FIG. 1A, a screw conveyor extraction system 100 comprising an extraction tank 105 configured with a varied geometry screw conveyor 104, in this example is inclined, is coupled to a biomass input 101 at the proximal end 150 and a biomass output 102 at the distal end 160. Although FIG. 1A shows a single screw conveyor extraction system 100, the methods described herein also contemplate a plurality (e.g., two, three, four, or more) of screw conveyor extraction systems 100 arranged in parallel.

The extraction tank 105 has a central axis 180 going through the varied geometry screw conveyor's 104 spindle 181.The incline of the extraction tank 105 can be varied such that the central axis 180 makes an incline angle θ 183 of about 1° to about 90° C. (e.g., about 3° to about) 15°) relative to a horizontal plane 182 intersecting the central axis 180. Varied geometry screw conveyor 104 is coupled to a sonication device 107 situated in close proximity (e.g., about 1 to about 10 cm) from a miscella drain 108. A solvent level 103 is highest at the proximal end 150 with the sonication device 107 and miscella drain 108 and lowest at the distal end 160 with the biomass output 102. The sonication device 107 is situated above the miscella drain 108 which is above the proximal end 150 of the varied geometry screw conveyor 104, preferably above the shaft of the proximal end 150 of the varied geometry screw conveyor 104. The varied geometry screw conveyor 104 has steadily decreasing pitch between the blades as to configured to compress the biomass as it passes from the biomass input at the proximal end 150 to the biomass output 102 at the distal end 160. The miscella drain 108 is fluidly coupled to a miscella stream output 109, which is, in turn, fluidly coupled to a membrane pump 111 that pressurizes the miscella stream and sends it through a membrane system 170, which may comprise 1, 2, 3, or 4 membranes, and be arranged as multiple membrane systems in parallel. Prior to the membrane system, the miscella stream may be filtered 110. The filtration system may comprise 1, 2, 3, 4, or 5 filters with about a 0.01-10 micrometer mesh size. These filters may be used to remove any large particular matter that is taken up from the miscella drain 108 in the extraction tank 105. The first membrane 120 is a dewaxing membrane that removes lipids from the miscella stream to give a first retentate 121 and a first permeate. The first permeate is transported via conduit 171 to desolvent membrane 130 that removes cannabinoids from the first permeate to give a second retentate, which can be called a “cannabinoid extract,” 131 and a second permeate. The second retentate may have any remaining solvent removed, for example, by a solvent removal means (e.g., evaporator or distillation unit 190) to produce a polished distillate 191 comprising cannabinoids and substantially no solvent. The second permeate can be collected in a solvent reserve tank 140 that can comprise a thermostat control. Solvent reserve tank 140 is fluidly coupled to the membrane system, and specifically to desolvent membrane 130, via conduit 172. Solvent reserve tank 140 collects second permeate from desovlent membrane 130, stores it, adjusts the aqueous solution temperature as necessary, and is coupled to a solvent return pump 141 coupled to a solvent return line 142 configured to send the recovered solvent into the extraction tank 105. The solvent may be filtered to remove any contaminants, including filtered through a membrane system to remove water or residual terpenes.

In sum, the system and methods described herein provide an extraction platform employing various solvents (e.g., alcohol, water, olive oil) where a pretreated Cannabis biomass input (e.g., chipped, stemmed) is fed into an extraction system (e.g., extraction system 100) comprising an inclined screw conveyor 104 into which a suitable solvent is provided to a suitable solvent level 103. The biomass can be pretreated by admixing with a solvent prior to being fed into the extraction system. For example, the biomass can be pretreated with solvent for any suitable amount of time, such as for about 1 minute to about 60 minutes, prior to being fed into the extraction system. The biomass is fed into extraction system 100 via biomass input 101 at the proximal end 150 of extraction system 100. Screw conveyor 104 moves the biomass through the solvent at an extraction rate based on the scale of the biomass input rate as a function of temperature and solvent-biomass contact time. In addition, sonication device 107 capable of various output frequencies is placed near the biomass input 101 to facilitate an expedient extraction and stimulate a biomass particle void in the solution near miscella drain 108.

Screw conveyor 104 has a progressive decrease in screw pitch with respect to the input (widest screw pitch, e.g., in zone one 161) to the output (progressively shorter screw pitch, e.g., in zone two 162), e.g., varied geometry screw conveyor. The change in screw pitch compresses the biomass thereby extracting solvent comprising desired compounds, the solvent flowing toward proximal end 150. Additionally, the extraction tank may have means to allow the solvent to flow back to the proximal end 150, e.g., trench, tube, or slots. The solvent may also be pumped from the distal end 160 to the proximal end 150, optionally filtered prior to re-introduction in the extraction tank.

At distal end 160 compressed biomass material is ejected through biomass output 102 into either a basin (not shown) or an additional conveyor system (not shown) to remove spent biomass material from screw conveyor extraction system 100. The spent biomass could be collected for further processing, composted, or discarded. The spent biomass may be moved via a screw conveyor, optionally a varied geometry screw conveyor, to have any residual solvent removed, and preferably collected for resuse.

Miscella drain 108 is located at proximal end 150 of screw conveyor 104 with an sonication device 107 in proximity to the miscella drain 108 in order to stimulate a particle “void” in solution. This “void” is created in order to prevent biomass particle buildup near the drain port and minimize filtration of the miscella post-extraction. In one embodiment, the sonication device 107 and the miscella drain 108 are placed above the proximal end 150 of screw conveyor 104. In an embodiment, the miscella drain 108 may be coupled to an extension means, optionally a pipe that may be capable of being moved to different positions in the extraction tank. In addition, while not wishing to be bound by any specific theory, it is believed that the use of a solvent in conjunction with ultrasonic power and frequency settings of the sonication device 107 disrupts trichomes without substantially disrupting Cannabis plant material. This has an unexpected advantage of only releasing the trichomes from the buds and leaving most of the Cannabis plant material intact. This avoids the problem of fibers, lignin, organelles, and other plant parts from entering the downstream processing steps (e.g., at the double membrane system 170). Further the method may be practiced at relatively high temperatures, e.g., −25° C. to 25° C., in contrast with the art that relies on much lower temperatures, e.g., −40° C. or lower. This results in a massive cost savings for heating and cooling of the system.

The second retentate may have any residual solvent removed. The system may comprise means for solvent removal 190 coupled to the membrane system 170 to remove solvent from the second retentate. The means for solvent removal may be a distiller, rotary evaporator, open evaporator, reduced pressure evaporator, rising film evaporator, falling film evaporator, chromatography column, fractionating column, distillation column, or a combination thereof. For example, a rising film evaporator, falling film evaporator, rotary evaporator, or other distillation equipment 190 may be coupled to the membrane system 170.

The miscella stream is pumped through a series of filters 110 to remove residual post-extraction particles. The miscella stream may be passed through a filter between the extraction tank and membrane system 170. The filter may have a size cutoff between 0.01 and 10 micrometers. The filter size cutoff may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 micrometers. The filtered miscella discharge is pumped through a membrane in double membrane system 170. As mentioned herein, first membrane 120 is a dewaxing membrane that removes lipids from the miscella stream to give a first retentate and a first permeate. The first permeate is transported via conduit 171 to desolvent membrane 130 that removes cannabinoids from the first permeate to give a second retentate and a second permeate. First membrane 120 in this series is selected by pore size to remove any materials larger than the selected membrane pore such as lipids. The desired extract molecule and solvent (first permeate) will permeate this first stage, leaving behind a first retentate, and be separated by a second-stage membrane (desolvent membrane 130) in the same manner to form a second retentate and a second permeate, as described herein. The first retentate can be sent for further post-processing and the second permeate can be thermostatically prepared (e.g., at solvent reserve tank 140) at the selected extraction temperature and returned to extraction tank 105 in proximity to biomass input 101.

Making reference to FIG. 1B, a screw conveyor extraction system 100 comprising an extraction tank 105 configured with a varied geometry screw conveyor 104, in this example is inclined, is coupled to a biomass input 101 at the proximal end 150 and a biomass output 102 at the distal end 160. Although FIG. 1B shows a single screw conveyor extraction system 100, the methods described herein also contemplate a plurality (e.g., two, three, four, or more) of screw conveyor extraction systems 100 arranged in parallel.

The extraction tank 105 has a central axis 180 going through the varied geometry screw conveyor's 104 spindle 181.The incline of the extraction tank 105 can be varied such that the central axis 180 makes an incline angle θ 183 of about 1° to about 90° C. (e.g., about 3° to about 15°) relative to a horizontal plane 182 intersecting the central axis 180. Varied geometry screw conveyor 104 is coupled to a sonication device 107 situated in close proximity (e.g., about 1 to about 10 cm) from a miscella drain 108. A solvent level 103 is highest at the proximal end 150 with the sonication device 107 and miscella drain 108 and lowest at the distal end 160 with the biomass output 102. The sonication device 107 is situated above the miscella drain 108 which is above the proximal end 150 of the varied geometry screw conveyor 104, preferably above the shaft of the proximal end 150 of the varied geometry screw conveyor 104. The varied geometry screw conveyor 104 has steadily decreasing pitch between the blades as to configured to compress the biomass as it passes from the biomass input at the proximal end 150 to the biomass output 102 at the distal end 160. The miscella drain 108 is fluidly coupled to a miscella stream output 109, which is, in turn, fluidly coupled to a membrane pump 111 that pressurizes the miscella stream and sends it through a single membrane system 170. The miscella stream output 109 may have a circulation pump in-line (not shown) to pressurize the miscella stream. Prior to the membrane system, the miscella stream may be filtered 110. The filtration system may comprise 1, 2, 3, 4, or 5 filters with about a 0.01-10 micrometer mesh size. These filters may be used to remove any large particular matter that is taken up from the miscella drain 108 in the extraction tank 105. The membrane 125 is a desolvent membrane that removes solvent from the miscella stream to give a retentate 121 and a permeate. The permeate is transported via conduit 172 to a solvent reserve tank 140 that can comprise a thermostat control. Solvent reserve tank 140 is fluidly coupled to the membrane system, and specifically to desolvent membrane 125, via conduit 172. Solvent reserve tank 140 collects permeate from desovlent membrane 125, stores it, adjusts the aqueous solution temperature as necessary, and is coupled to a solvent return pump 141 coupled to a solvent return line 142 configured to send the recovered solvent into the extraction tank 105. The solvent may be filtered to remove any contaminants, including filtered through a membrane system to remove water or residual terpenes.

The membrane system may comprise three membranes (not shown in FIG. 1). The membranes may be organic solvent stable membranes, for example silicone membranes or modified polyimide membranes. For example, in a three membrane system, the first membrane may be a dewaxing membrane with a molecular weight cutoff of about 500 Da, the second membrane may be a desolvent membrane with a molecular weight cut off of about 300 Da, and he third membrane may be a dewatering membrane with a molecular cutoff between about 20 Daltons and 40 Daltons. This allows the third membrane to be used as a dewatering membrane to remove any residual water from the solvent.

### Cannabis Plant Material

The starting material that is provided to biomass input 101 for the methods and systems described herein can be any Cannabis plant material, including but not limited to raw Cannabis plant material, Cannabis extracts produced by exposing Cannabis plant material to a solvent, pretreated Cannabis plant material, parts of any Cannabis plant, or combinations thereof. The starting material may also be hemp (industrial hemp), e.g., Cannabis with less than 0.3% THC by dry weight.

### Processing of Cannabis Plant Material

For pretreatment, the Cannabis plant material can be washed, dried, and/or comminuted. The Cannabis plant material can be added to the screw conveyor extraction system 100 after being comminuted and mixed with a solvent for a suitable amount of time (e.g., 1-10 minutes) at any suitable temperature (e.g., −20° C. to 25° C.). It has been surprisingly found that the system and method described herein does not require pre-treatment before the Cannabis plant material is processed according to the methods described herein. This is in contrast with the state of the art that requires extensive pre-treatment, processing with organic solvents, and/or super critical carbon dioxide. The Cannabis plant material, whether pretreated or not, can be mixed directly with any suitable solvent (e.g., an alcohol) at any suitable ratio by weight of solvent to biomass to extract cannabinoids, lipids, and terpenes from the biomass. For example, the Cannabis plant material can be admixed with an alcohol (e.g., methanol) at a 5:1, solvent to biomass ratio by weight for about 1 minute to about 10 minutes at −20° C. to 25° C. The Cannabis plant material can then be introduced into screw conveyor extraction system 100 by means of a screw conveyor and subjected to ultrasonic cavitation. The ultrasonic cavitation can serve to, among other things, disrupt phospholipid structures in which cannabinoids are concentrated. It has been surprisingly discovered that directly applying ultrasonic cavitation to the Cannabis plant material in an alcohol can release over 95% of cannabinoids contained therein in less than 5 minutes. This contrasts with current methods that require hours and release less than 50% of the cannabinoids.

Although the examples described herein use a screw conveyor extraction system 100, other systems can be used. Regardless of the system used, the Cannabis plant material can be admixed with a suitable solvent (e.g., an alcohol), subjected to ultrasonic cavitation, and maintained at a temperature between −40° C. and 30° C., where appropriate. The ultrasonic cavitation can serve to, among other things, disrupt the phospholipid structures on the trichomes on Cannabis. This releases the cannabinoids into the solvent. The plant material, comprising cellulose, hemicellulose, and lignin, is moved away from the proximal end of the screw conveyor processor by means of the screw conveyor. In the example provided in FIG. 1, varied geometry screw conveyor 104 moves the processed Cannabis plant material away from the ultrasonic cavitation device (e.g., sonication device 107), and progressively applies pressure by means of tighter screw spacing, until, at the distal end, the processed Cannabis plant material is expelled from the screw conveyor processor. The process Cannabis material can be dried to remove the residual alcohol, recovered for further processing, or discarded.

Suitable solvents that can be used to extract cannabinoids from the Cannabis plant material include organic solvents, water (e.g., potable water), and combinations thereof. Organic solvents include alcohols, ethers, esters, ketones, alkanes, and combinations thereof. Examples of alcohols include methanol, ethanol, n-propanol, 1-propanol, n-butanol, sec-butanol, t-butanol, 1-pentanol, amyl alcohol, isoamyl alcohol, 1-hexanol, 2-hexanol, 3-hexanol, 1-heptanol, 2-heptanol, 3-heptanol, 4-heptanol, 1-octanol, 2-octanol, 1-nonanol, 1-decanol, or a mixture thereof. For example, the alcohol can be methanol, ethanol, or a mixture thereof. The solvent can be ethanol (e.g., as the sole solvent). The solvent can be methanol (e.g., as the sole solvent). The solvent can also comprise water. Examples of ethers include diethyl ether, dipropyl ether, tetrahydrofuran, and the like and combinations thereof. Examples of esters include ethyl acetate and the like. Examples of ketones include acetone, methyl ethyl ketone, and the like and combinations thereof. Examples of alkanes include pentane, hexane, heptane, and the like and combinations thereof.

Suitable solvents can be substantially free of detergents, absorbing agents, plasticizers, emulsifiers, solubilizers, organic solvents, or any combination thereof.

### Ultrasonic Cavitation

A processing unit (e.g., extraction system 100) comprising an ultrasonic cavitation device (e.g., sonication device 107), a screw conveyor, biomass input port, biomass output port, and drain. The processing unit can be an alcohol-containing tank that has at least one ultrasonic cavitation device comprising an ultrasonic transducer. The ultrasonic transducer can be of any suitable shape and dimension. For example, the ultrasonic probe can have any suitable shape (e.g., horn, probe, cup, or rod shaped) and can be immersed in a fluid or attached to the inside of the processing unit. Although conventional transducers are mounted to the outside of the tank, the ultrasonic transducer described herein can be disposed internal to the tank and suspended in the solvent column by one or more support brackets. Generally, the submerged transducers are more efficient, and the external transducer tend to damage the ultrasonic tank over time. The transducer can be a rod made of titanium or other metal, or metal alloy. The transducer can have a length of one to four feet long and can be rod shaped or any other suitable shape (e.g., horn, probe or cup shaped). The ultrasonic probe may be place above the miscella drain which is above the proximal end of the screw conveyor. In another embodiment, the miscella drain may be position parallel to two sonication devices in the extraction tank above the screw conveyor. As the biomass is fed into the system, it is subjected to ultrasonic cavitation that releases the trichomes, leaving the biomass mostly intact. This avoids the problem of disrupting the lignocellulosic biomass, releasing nucleic acids, or bursting plant cells. The inventor surprising found that the ultrasonic conditions disrupted the trichomes where the majority of the biomass sinks with a miscella above the heavier sonicated biomass. This allows for the easy collection of the miscella by means of a miscella drain and have the heavier biomass be moved away from the sonication device.

The ultrasonic energy created by the transducer produces sufficient intensity of ultrasonic energy to disrupt the trichome structure, releasing the cannabinoids into a miscella stream. An electrical signal from a generator feeds into the transducers, creating sound in the fluid strong enough to create cavitation of the solution and disrupt the trichomes. The electrical signal can come from a generator located external to the tank, and it can be connected to the transducers located within the tank by a lead. In contrast with current methods that focus on disrupting the plant material, which leads to the release of fibers, lignin, organelles, nucleic acids that tend to foul membranes, a synergistic combination of ultrasonic power and frequency in a solvent at a short time interval releases the trichomes, while leaving the Cannabis plant material substantially intact, has been discovered. As discussed herein, this synergistic combination of the ultrasound settings and warm alcohol solvent (e.g., methanol) is successful in releasing trichomes from hemp material that was about 8-10 months old post-harvest where the trichome lipids had begun to polymerize making them resistant to cannabinoid extraction.

Temperature

The solution and/or the mixture (e.g., a mixture comprising Cannabis plant material and at least one solvent) inside the processing unit (e.g., extraction system 100) comprising the Cannabis plant material can undergo ultrasonic cavitation at any suitable temperature. The temperature can be between about −25° C. to about 25° C., such as from about −10° C. to about 10° C. For example, when the solvent used in the processing unit is ethanol, the temperature can be below the flash point of ethanol (e.g., about 16° C.). In other examples, the temperature of the solution and/or the mixture inside the processing unit can be about −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. In other examples, the temperature of the solution and/or the mixture inside the processing unit can be between about −10° C. to 10° C., −10° C. to 0° C., −15° C. to 20° C., or −15° C. to 5° C.

The temperature in the processing unit (e.g., extraction system 100) can be maintained by thermal insulation means.

Time

The Cannabis plant material/solvent mixture may undergo ultrasonic cavitation, where ultrasonic cavitation is applied for any suitable amount of time, such as from about 1 second to about 60 seconds, about 20 to about 40 seconds or the Cannabis plant material can be undergo ultrasonic treatment, where ultrasonic cavitation is applied for about 30 seconds. The Cannabis plant material can undergo ultrasonic treatment, where ultrasonic cavitation is applied for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The Cannabis plant material can be exposed to ultrasonic energy, where ultrasonic cavitation is applied for 1-50 seconds, 5-50 seconds, 10-50 seconds, 25-35 seconds, 21-45 seconds, 25-40 seconds, 30-40 seconds, 27-45 seconds, 29-39 seconds, 21-31 seconds, 23-32 seconds, 29-38 seconds, or 24-36 seconds.

The Cannabis plant material/solvent mixture may undergo ultrasonic cavitation, where ultrasonic cavitation is applied for any suitable amount of time, such as from about 1 to about 10 minutes, e.g., where ultrasonic cavitation is applied for about 3 minutes. The Cannabis plant material can undergo ultrasonic treatment, where ultrasonic cavitation is applied for about 1, 2, 3, 4, 5, 6, 7, 8, 9, or 10 minutes. The Cannabis plant material can be exposed to ultrasonic energy, where ultrasonic cavitation is applied for 1-5 minutes, 5-10 minutes, 1-7 minutes, 2-5 minutes, 1-4 minutes, 2-4 minutes, 3-4 minutes, 2-3 minutes, or 1-3 minutes. The Cannabis material may be subjected to ultrasonic cavitation for about 3 minutes contact time.

The ultrasonic cavitation may be applied in pulses. The ultrasonic cavitation may be pulsed, i.e., applied via a mixture of on and off cycles. The ultrasonic cavitation pulsing “on/off” cycle may be applied for 5 seconds, then off for 5 seconds, and repeated for a total of 30 to 240 seconds. The ultrasonic cavitation may be applied for 5 seconds on/5 seconds off, 10 seconds on/10 seconds off, 15 seconds on/15 seconds off, 20 seconds on/20 seconds off, 25 seconds on/25 seconds off, 30 seconds on/30 seconds off, 35 seconds on/35 seconds off, 40 seconds on/40 seconds off, 45 seconds on/45 seconds off, or 50 seconds on/50 seconds off. The ultrasonic cavitation may be applied for 5 seconds on/10 seconds off, 10 seconds on/20 seconds off, 25 seconds on/15 seconds off, 20 seconds on/40 seconds off, 35 seconds on/25 seconds off, 35 seconds on/30 seconds off, 135 seconds on/35 seconds off, 120 seconds on/30 seconds off, 60 seconds on/120 seconds off, or 100 seconds on/50 seconds off

For example, the ultrasonic cavitation may be applied for 5 seconds, then off for 5 seconds, and repeated for a total of 30 to 180 seconds. The ultrasonic cavitation may be an equal set of “on/off” pulses, for example, 5 seconds on/5 seconds off, 10 seconds on/10 seconds off, 15 seconds on/15 seconds off, 20 seconds on/20 seconds off, 25 seconds on/25 seconds off, 30 seconds on/30 seconds off, 35 seconds on/35 seconds off, 40 seconds on/40 seconds off, 45 seconds on/45 seconds off, or 50 seconds on/50 seconds off. In another embodiment, the ultrasonic cavitation may be an unequal set of “on/off” pulses, for example, 5 seconds on/10 seconds off, 10 seconds on/20 seconds off, 25 seconds on/15 seconds off, 20 seconds on/40 seconds off, 35 seconds on/25 seconds off, 35 seconds on/30 seconds off, 135 seconds on/35 seconds off, 120 seconds on/30 seconds off, 60 seconds on/120 seconds off, or 100 seconds on/50 seconds off.

Power

Any high-power ultrasonic device can be used in the methods and systems described herein. Power requirements are a function of the amount of biomass:solvent mixture being subjected to ultrasonic cavitation. The power range can be expressed in terms of kilowatts of ultrasonic energy per kilogram of biomass and solvent, for example the ultrasonic cavitation can be applied at a power of from about 0.1 to about 1 kW/kg of biomass and solvent by weight (e.g., Cannabis plant material and at least one solvent by weight), which is an amount of power sufficient to disrupt the trichomes. For example, the ultrasonic cavitation can be applied at a power of about 0.1, 0.2, 0.3, 0.4, 0.5, 0.6, 0.7, 0.8, 0.9, or about 1.0 kW/kg of biomass and solvent by weight. The ultrasonic cavitation can be applied at a power of about 0.1 to about 0.9 kW/kg of biomass and solvent by weight, about 0.5 to about 1.0 kW/kg of biomass and solvent by weight, about 0.25 to about 0.75 kW/kg of biomass and solvent by weight, about 0.3 to about 0.6 kW/kg of biomass and solvent by weight, about 0.2 to about 0.8 kW/kg of biomass and solvent by weight, or about 0.15 to about 0.95 kW/kg of biomass and solvent by weight.

Frequency

The ultrasonic unit can use ultrasonic energy at about 1 to about 100 kHz. For example, the ultrasonic system can use a frequency of about 35 kHz. The generator should produce enough power to generate a transducer frequency about 1 kHz to about 100 kHz. For example, the ultrasonic cavitation can be performed at a frequency of about 1 kHz, 2 kHz, 3 kHz, 4 kHz, 5 kHz, 6 kHz, 7 kHz, 8 kHz, 9 kHz, 10 kHz, 11 kHz, 12 kHz, 13 kHz, 14 kHz, 15 kHz, 16 kHz, 17 kHz, 18 kHz, 19 kHz, 20 kHz, 21 kHz, 22 kHz, 23 kHz, 24 kHz, 25 kHz, 26 kHz, 27 kHz, 28 kHz, 29 kHz, 30 kHz, 31 kHz, 32 kHz, 33 kHz, 34 kHz, 35 kHz, 36 kHz, 37 kHz, 38 kHz, 39 kHz, 40 kHz, 41 kHz, 42 kHz, 43 kHz, 44 kHz, 45 kHz, 46 kHz, 47 kHz, 48 kHz, 49 kHz, 50 kHz, 51 kHz, 52 kHz, 53 kHz, 54 kHz, 55 kHz, 56 kHz, 57 kHz, 58 kHz, 59 kHz, 60 kHz, 61 kHz, 62k Hz, 63 kHz, 64 kHz, 65 kHz, 66 kHz, 67 kHz, 68 kHz, 69 kHz, 70 kHz, 71 kHz, 72 kHz, 73 kHz, 74 kHz, 75 kHz, 76 kHz, 77 kHz, 78 kHz, 79 kHz, 80 kHz, 81 kHz, 82 kHz, 83 kHz, 84 kHz, 85 kHz, 86 kHz, 87 kHz, 88 kHz, 89 kHz, 90 kHz, 91 kHz, 92, kHz, 93 kHz, 94 kHz, 95 kHz, 96 kHz, 97 kHz, 98 kHz, 99 kHz, or about 100 kHz. The ultrasonic cavitation can be at a frequency of about 20 to about 50 kHz, about 10 to about100 kHz, about 30 to about 70 kHz, about 280 to about 40 kHz, about 21 to about 450 kHz, about 26 to about 380 kHz, about 29 to about 39 kHz, about 32 to about 36 kHz, about 34 to about 38 kHz, about 25 to about 40 kHz, about 30 to about 40 kHz, about 10 to about 40 kHz, about 30 to about 45 kHz, about 25 to about 50 kHz, about 31 to about 39 kHz, or about 32 to about 42 kHz. The ultrasonic treatment can be at a frequency of about 35 kHz. The ultrasonic treatment can be performed at a frequency of about 35 kHz, at about 80 Watts of power, for about 30 seconds. The ultrasonic treatment can be performed at a frequency of about 35 kHz, at about 80 Watts of power, for about 3 minutes.

### Cannabinoids

Cannabinoids extracted into the solvent include tetrahydrocannabinol, cannabidiol (CBD), cannabigerol, cannabinol, cannabichromene, cannabigerivarin, tetrahydrocannabivarin, cannabidivarin, cannabichromevarin, or a mixture thereof. It should be appreciated that the extract may also include cannabinoid acids, such as cannabigerolic acid, Δ9-tetrahydrocannabinolic acid (THC), Δ8-tetrahydrocannabinolic acid (THC), cannabidiolic acid, cannabichromenenic acid, cannabigerovarinic acid, tetrahydrocanabivarinic acid, cannabidivarinic acid, cannabichromevarinic acid, or a mixture thereof. The cannabinoids may be collected in a Cannabis extract and further processed.

The concentration of a cannabinoid in a cannabinoid extract (e.g., second retentate) may range between about 0% (w/w) and about 50% (w/w), between about 5% (w/w) and about 45% (w/w), between about 10% (w/w) and about 40% (w/w), between about 15% (w/w) and about 35% (w/w), or between about 20% (w/w) and about 30% (w/w), inclusive. The cannabinoid concentration in a cannabinoid extract (e.g., second retentate) or miscella stream also includes ranges of between about 0% (w/w) and about 5% (w/w), between about 5% (w/w) and about 10% (w/w), between about 10% (w/w) and about 15% (w/w), between about 15% (w/w) and about 20% (w/w), between about 20% (w/w) and about 25% (w/w), between about 25% (w/w) and about 30% (w/w), between about 30% (w/w) and about 35% (w/w), between about 35% (w/w) and about 40% (w/w), between about 40% (w/w) and about 45% (w/w), or between about 45% (w/w) and about 50% (w/w), inclusive. Therefore, the concentration of the cannabinoid a cannabinoid extract (e.g., second retentate) can be between about 0% (w/w) and about 5% (w/w), between about 5% (w/w) and about 10% (w/w), between about 10% (w/w) and about 15% (w/w), between about 15% (w/w) and about 20% (w/w), between about 20% (w/w) and about 25% (w/w), between about 25% (w/w) and about 30% (w/w), between about 30% (w/w) and about 35% (w/w), between about 35% (w/w) and about 40% (w/w), between about 40% (w/w) and about 45% (w/w), between about 45% (w/w) and about 50% (w/w), between about 50% (w/w) and about 95% (w/w), between about 60% (w/w) and about 90% (w/w), between about 70% (w/w) and about 90% (w/w), between about 80% (w/w) and about 99% (w/w), between about 80% (w/w) and about 95% (w/w), between about 80% (w/w) and about 90% (w/w), between about 75% (w/w) and about 85% (w/w), inclusive. These ranges may also apply to the amount of cannabinoids in the miscella stream.

Terpenes isolated using the systems and methods described herein include but are not limited to alpha bisabolol, alpha phellandrene, alpha pinene, beta caryophyllene, beta pinene, cadinene, camphene, camphor, citral, citronellol, delta 3 carene, eucalyptol, eugenol, gamma terpinene, geraniol, humulene, limonene, linalool, nerol, nerolidol, ocimene, para-cymene, phytol, pulegone, terpineol, terpinolene, valencene, or mixtures thereof. The terpenes may be collected and used in further processing.

### Filtration System

After the Cannabis plant material has been subjected to ultrasonic cavitation in a solvent as described herein, one obtains a miscella stream comprising a solvent comprising a mixture of lipids and cannabinoids. This miscella stream may be subjected to filtration to remove any particulate matter that passes through the miscella drain in the extraction tank. The miscella stream may be passed through a filter before being subject to membrane filtration. The filtration system may comprise 1, 2, 3, 4, 5, or 6 filters with about a 0.01-10 micrometer mesh size. For example, the filters may have a 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 micrometer mesh size. The filter size cutoff may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 micrometers. The filter may filter out plant material, mold spores, or other contaminants. The miscella stream, after filtration, may be referred to as a crude extract. This crude extract comprising cannabinoids, solvent, and lipids, may be collected. Optionally, the crude extract comprising cannabinoids, solvent, and lipids may be removed (e.g., winterized), for example, by means of a membrane system.

### Membrane Systems

After the Cannabis plant material has been subjected to ultrasonic cavitation in a solvent as described herein, one obtains a miscella stream comprising a solvent, lipids, and cannabinoids. The miscella stream is collected by means of the miscella drain 108 and, is optionally pumped through a filtration system described herein, before being passed through a membrane system 170. The membrane system may be a single, double, or triple membrane system.

In a single membrane system 170, the first membrane is a dewatering membrane that removes substantially all of the solvent from the crude extract. This crude extract may be collected, and, optionally be substantially free of lipids (e.g., winterized).

In a double membrane system 170, the first membrane 120 is a dewaxing membrane that removes lipids from the miscella stream to give a first retentate and a first permeate. The first permeate is transported via conduit 171 to a second, desolvent membrane 130 that removes cannabinoids from the first permeate to give a second retentate and a second permeate. This cannabinoid extract substantially free of lipids may be referred to as a cannabinoid distillate. The cannabinoid distillate may be subjected to solvent extract means to polish off any remaining solvent.

In a triple membrane system 170, the first membrane 120 is a dewaxing membrane that removes lipids from the miscella stream to give a first retentate and a first permeate. The first permeate is transported via conduit 171 to a second, desolvent membrane 130 that removes cannabinoids from the first permeate to give a second retentate and a second permeate. The second permeate, alternatively, both the second retentate and second permeate is transported via conduit to a third, dewatering membrane that removes any residual solvent, optionally contaminants, e.g., water. This cannabinoid extract may be referred to as a refined distillate.

Dewaxing Membrane

A solution, optionally the miscella stream, may be filtered through a dewaxing membrane to produce a first permeate and a first retentate. Suitable dewaxing membranes have a molecular weight cutoff of about 150 Da, 175 Da, 200 Da, 225 Da, 250 Da, 275 Da, 300 Da, 325 Da, 350 Da, 350 Da, 375 Da, 400 Da, 425 Da, 450 Da, 500 Da, 550 Da, 600 Da, 650 Da, 700 Da, 750 Da, 800 Da, 850 Da, 900 Da, 950 Da, or 1,000 Da. Other suitable first membrane filters may have a molecular weight cutoff of about 500 Da, 900 Da, or 1,000 Da. The first retentate may comprise lipids. The second permeate comprises the solvent and cannabinoids.

The membrane may be an organic solvent stable membrane. For example, the membrane may be a silicone membrane, polymeric organophilic membrane, polyimide membrane, including modified polyimide membrane, polyflouride, polyether-ether ketone, methyl-ethyl ketone (MEK), polyethylene, polypropylene, cellulose acetate, polystyrene, polytetrafluoroethylene, polyimide, or polysilane membrane. Suitable membranes for use in the disclosed methods may include but are not limited to: Synder NFG, Synder XT, Synder MT, and Synder VT produced by Synder Filtration, Inc. (Vacaville, Calif.); GE Osmonics UF GK, GE Osmonics UF GH, GE Osmonics UF PT, and GE Osmonics UF GE available from SterlitechCorporation (Kent, Wash.); TriSep OF UA60, TriSep NF XN45, and TriSep NF TS40 produced by TriSep Corporation (Goleta, Calif.); and Dow Filmtec NF produced by Dow Chemical Company (Midland, Mich.) Organic solvent stable filters, such as SolSep UF10706, SolSep UF03705, and SolSep NF080105 produced by SolSep BV (St. Eustatius, Netherlands); DuraMem produced by Evonik (Hesse, Germany) and Novamem PVDF20 and Novamem PEEK 1000 produced by Novamen Ltd. (Schlieren, Switzerland) can be used. A suitable membrane for the first membrane filtration step can be a DuraMem 500 produced by Evonik (Hesse, Germany). Because the molecular weight of cannabinoids is about 314 g/mole, the cannabinoids pass through the first membrane into the first permeate, which can also include terpenes. Gums and lipids, such as fats, waxes, phospholipids, and fatty acids may remain in the first retentate stream. The first retentate can be discarded, recycled to extract further cannabinoids, terpenes, or a mixture thereof, or collected for further processing.

Desolvent Membrane

A solution, for example, the first permeate may be filtered through a desolvent membrane 130, to produce a permeate and a retentate. Suitable desolvent membranes may have a molecular weight cutoff of about 100 Da, 200 Da, or 300 Da. The retentate may comprise cannabinoids (and terpenes). The permeate may comprise the solvent.

The desolvent membrane may be an organic solvent stable membrane. For example, the desolvent membrane may be a silicone membrane, polymeric organophilic membrane, polyimide membrane, including modified polyimide membrane, polyflouride, polyether-ether ketone, methyl-ethyl ketone (MEK), polyethylene, polypropylene, cellulose acetate, polystyrene, polytetrafluoroethylene, polyimide, or polysilane membrane. The desolvent membrane may have a molecular weight cutoff from about 10 Da to about 100 Da, from about 25 Da to about 95 Da, or from about 50 Da to about 90 Da. The desolvent membrane can have a molecular weight cutoff from about 50 Da to about 100 Da, from about 20 Da to about 50 Da, from about 75 Da to about 100 Da, from about 25 Da to about 50 Da, or from about 75 Da to about 95 Da. The desolvent membrane can have a molecular weight cutoff of about 25 Da, 30 Da, 45 Da, 50 Da, 75 Da, 100 Da, 125 Da, 150 Da, 175 Da, 200 Da, 225 Da, 250 Da, 275 Da, 300 Da, 325 Da, 350 Da, 375 Da, 400 Da, or 425 Da.

For example, desolvent membranes may include but are not limited to Synder NFG, Synder XT, and Synder NFX produced by Synder Filtration, Inc. (Vacaville, Calif.); GE Osmonics UF GE, GE Osmonics UF Duracid, and GE Osmonics UF DK available from Sterlitech Corporation (Kent, Wash.); TriSep NF TS80 and TriSep NF XN45 produced by TriSep Corporation (Goleta, Calif.); Dow Filmtec NF produced by Dow Chemical Company (Midland, Mich.); DuraMem 500 produced by Evonik (Essen, Germany), and Nanostone NF NF4 and Nanostone NF NF8 produced by Nanostone Water Inc. (Eden Prairie, Minn.). Surprisingly, such membranes maintained their integrity when used to with ethanol and/or methanol without dilution with water. Suitable organic solvent stable membranes include, SolSep NF090801, SolSep NF03705, SolSep SR1 NF080105, SolSep UF10706, SolSep UF03705, SolSep NF08105, and SolSep NF10706 produced by SolSep BV (St. Eustatius, Netherlands); and Novamem PVDF20 and Novamem PEEK 1000 produced by Novamen Ltd. (Schlieren, Switzerland). Silicone or modified polyimide membranes may also be used as a desolvent membrane.

Because the molecular weight of cannabinoids is about 314 g/mole, the cannabinoids pass through the first membrane into the first permeate, which also includes terpenes, and are collected at the second membrane as the second retentate. The second retentate comprising the cannabinoids can be collected for further processing. The second permeate comprising the solvent can be collected and reused in the extraction process. In some embodiments, the collected solvent in the second permeate may undergo further membrane filtration to remove possible contaminants.

Dewatering Membrane

A solution may be filtered through a dewatering membrane to produce a permeate and a retentate. Suitable dewatering membranes have a molecular weight cutoff of about 20-30 Da. The retentate may comprise cannabinoids (and terpenes). The permeate comprises the solvent.

Suitable dewatering membranes include organic solvent soluble membranes. The dewatering membrane may be a silicone membrane, polymeric organophilic membrane, polyimide membrane, including modified polyimide membrane, polyflouride, polyether-ether ketone, methyl-ethyl ketone (MEK), polyethylene, polypropylene, cellulose acetate, polystyrene, polytetrafluoroethylene, polyimide, or polysilane membrane. The dewatering membrane may have a molecular weight cutoff from about 10 Da to about 40 Da, from about 25 Da to about 35 Da, or from about 20 Da to about 30 Da. The second membrane can have a molecular weight cutoff from about 21 Da to about 31 Da, from about 20 Da to about 35 Da, from about 25 Da to about 30 Da. The second membrane can have a molecular weight cutoff of about 20 Da, 21 Da, 22 Da, 23 Da, 24 Da, 25 Da, 26 Da, 27 Da, 28 Da, 29 Da, 30 Da, 31 Da, 32 Da, or 33 Da.

For example, dewatering membranes may include but are not limited to Synder NFG, Synder XT, and Synder NFX produced by Synder Filtration, Inc. (Vacaville, Calif.); GE Osmonics UF GE, GE Osmonics UF Duracid, and GE Osmonics UF DK available from Sterlitech Corporation (Kent, Wash.); TriSep NF TS80 and TriSep NF XN45 produced by TriSep Corporation (Goleta, Calif.); Dow Filmtec NF produced by Dow Chemical Company (Midland, Mich.); DuraMem 500 produced by Evonik (Essen, Germany), and Nanostone NF NF4 and Nanostone NF NF8 produced by Nanostone Water Inc. (Eden Prairie, Minn.) Suitable organic solvent stable membranes include, SolSep NF090801, SolSep NF03705, SolSep SR1 NF080105, SolSep UF10706, SolSep UF03705, SolSep NF08105, and SolSep NF10706 produced by SolSep BV (St. Eustatius, Netherlands); and Novamem PVDF20 and Novamem PEEK 1000 produced by Novamen Ltd. (Schlieren, Switzerland).

Because the molecular weight of methanol is about 32 g/mole and the molecular weight of water is about 18 g/mol, the water passes through the third membrane into the third permeate, and the solvent (e.g., methanol) is collected at the third membrane as the third retentate. The third retentate comprising the solvent can be collected for reuse. The third permeate comprising water can be collected.

In some embodiments, the collected solvent in the third permeate may undergo further membrane filtration to remove possible contaminants. The third permeate may be passed through a filter before being collected and/or reused. The filter may have a size cutoff between 0.01 and 10 micrometers. The filter size cutoff may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 micrometers. The filter may filter out contaminants.

Solvent Temperatures

In the methods disclosed herein, the temperature of the solvent, such as the temperature of the solvent as it enters a membrane, can range from about −40° C. to about 20° C., from about −30° C. to about 20° C., from about −20° C. to about 20° C., from about −10° C. to about 20° C., or from about 0° C. to about 10° C. These temperature ranges can also be expressed as from about −40° C. to about −20° C., from about −30° C. to about −20° C., from about −20° C. to about −10° C., from about −10° C. to about 0° C., from about 0° C. to about 10° C., from about 10° C. to about 20° C., from about 15° C. to about 20° C., from about 10° C. to about 15° C., or from about 4° C. to about 10° C. Thus, the temperature of the solvent is about −40° C., about −35° C., about −30° C., about −25° C., about −20° C., about −15° C., about −10° C., about −5° C., about 0° C., about 5° C., about 10° C., about 15° C., about 20° C., about 5° C., about 15° C., about 4° C., about 14° C., about −5° C., or about 5° C.

Solvent Pressures

The pressure of the solvent at a membrane may range from about 50 pound-force per square inch (psi) to about 600 psi, about 75 psi to about 500 psi, about 100 psi to about 400 psi, about 125 psi to about 300 psi, or about 150 psi to about 250 psi. Suitable operating pressure ranges can also be expressed as about 50 psi to about 100 psi, from about 100 psi to about 150 psi, from about 150 psi to about 200 psi, from about 200 psi to about 250 psi, from about 250 psi to about 300 psi, from about 300 psi to about 350 psi, from about 350 psi to about 400 psi, from about 400 psi to about 450 psi, or from about 450 psi to about 500 psi.

Volumetric Flow Rate of Solvent

The volumetric flow rate (Q) of the solvent at a membrane depends on the surface are of the membrane. For example, the volumetric flow rate of the solvent can be from about 0 L/h to about 1000 L/h, from about 10 L/h to about 750 L/h, from about 20 L/h to about 500 L/h, from about 30 L/h to about 450 L/h, from about 40 L/h to about 400 L/h, from about 50 L/h to about 350 L/h, from about 75 L/h to about 300 L/h, from about 100 L/h to about 250 L/h. In full scale processes in which the surface area of the membrane is greater than about 25 m2, the volumetric flow rate of the solvent may exceed 1000 L/h. The volumetric flow rate of the solvent may also range from about 0 L/h to about 10 L/h, from about 10 L/h to about 50 L/h, from about 50 L/h to about 100 L/h, from about 100 L/h to about 200 L/h, from about 200 L/h to about 400 L/h, from about 400 L/h to about 600 L/h, from about 600 L/h to about 800 L/h, from about 800 L/h to about 1000 L/h. Solvent volumetric flow rates include about 5 L/h, about 10 L/h, about 15 L/h, about 20 L/h, about 25 L/h, about 50 L/h, about 75 L/h, about 100 L/h, about 125 L/h, about 150 L/h, about 175 L/h, about 200 L/h, about 250 L/h, about 400 L/h, about 600 L/h, about 800 L/h, about 1000 L/h.

Permeate Temperature

The temperature of the permeates range from about −40° C. to about 20° C., from about −30° C. to about 20° C., from about −20° C. to about 20° C., from about −10° C. to about 20° C., or from about 0° C. to about 10° C. These temperature ranges can also be expressed as from about −40° C. to about −20° C., from about −30° C. to about −20° C., from about −20° C. to about −10° C., from about −10° C. to about 0° C., from about 0° C. to about 10° C., from about 10° C. to about 20° C., from about 15° C. to about 20° C., from about 10° C. to about 15° C., or from about 4° C. to about 10° C. Thus, the temperature of the permeate can be about −40° C., about −35° C., about −30° C., about −25° C., about −20° C., about −15° C., about −10° C., about −5° C., about 0° C., about 5° C., about 10° C., about 15° C., about 20° C., about 5° C., about 15° C., about 4° C., about 14° C., about −5° C., or about 5° C. The temperature of the permeate may be about −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C.

Pressures

The disclosed methods employ pressures of the permeates include ranges from about 50 pound-force per square inch (psi) to about 500 psi, about 75 psi to about 500 psi, about 100 psi to about 400 psi, about 125 psi to about 300 psi, or about 150 psi to about 250 psi. Other pressure ranges include about 50 psi to about 100 psi, from about 100 psi to about 150 psi, from about 150 psi to about 200 psi, from about 200 psi to about 250 psi, from about 250 psi to about 300 psi, from about 300 psi to about 350 psi, from about 350 psi to about 400 psi, from about 400 psi to about 450 psi, or from about 450 psi to about 500 psi.

Permeate Flow Rate

The flow rate of the permeates can be 0-1,000 liters per hour. Because the flow rate is proportional to the surface area of the membrane, the flow rate of the permeate may scale much higher. In other words, the flux of the permeate through a membrane ranges from about 0 L/h·m² to about 1000 L/h·m², from about 10 L/h·m² to about 750 L/h·m², from about 20 L/h·m² to about 500 L/h·m², from about 30 L/h·m² to about 450 L/h·m², from about 40 L/h·m² to about 400 L/h·m², from about 50 L/h·m² to about 350 L/h·m², from about 75 L/h·m² to about 300 L/h·m², from about 100 L/h·m² to about 250 L/h·m². The flux of the permeate through the membrane may also range from about 0 L/h·m² to about 10 L/h·m², from about 10 L/h·m² to about 50 L/h·m², from about 50 L/h·m² to about 100 L/h·m², from about 100 L/h·m² to about 200 L/h·m², from about 200 L/h·m² to about 400 L/h·m², from about 400 L/h·m² to about 600 L/h·m², from about 600 L/h·m² to about 800 L/h·m², from about 800 L/h·m² to about 1000 L/h·m². Particular fluxes within such ranges include about 5 L/h·m², about 10 L/h·m², about 15 L/h·m², about 20 L/h·m², about 25 L/h·m², about 50 L/h·m², about 75 L/h·m², about 100 L/h·m², about 125 L/h·m², about 150 L/h·m², about 175 L/h·m², about 200 L/h·m², about 250 L/h·m², about 400 L/h·m², about 600 L/h·m², about 800 L/h·m², about 1000 L/h·m².

Retentate Flow Rate

The flow rate of a retentate can be 0-50 liters per hour. The lipid rejection is greater than about 500 Daltons. Thus, the retentate may have a lipid concentration of from about 0% (w/w) to about 90% (w/w), from about 0.1% (w/w) to about 80% (w/w), from about 1% (w/w) to about 70% (w/w), from about 2% (w/w) to about 60% (w/w), from about 5% (w/w) to about 50% (w/w), from about 10% (w/w) to about 40% (w/w), from about 15% (w/w) to about 30% (w/w), or from about 20% (w/w) to about 25% (w/w). The first permeate lipid concentration may also range from about 0% (w/w) to about 0.1% (w/w), from about 0.1% (w/w) to about 1% (w/w), from about 1% (w/w) to about 2% (w/w), from about 2% (w/w) to about 5% (w/w), from about 5% (w/w) to about 10% (w/w), from about 10% (w/w) to about 15% (w/w), from about 15% (w/w) to about 20% (w/w), from about 20% (w/w) to about 30% (w/w), from about 30% (w/w) to about 40% (w/w), from about 40% (w/w) to about 50% (w/w), from about 50% (w/w) to about 70% (w/w), or from about 70% (w/w) to about 90% (w/w). Lipid concentration of less than about 90% (w/w), less than about 80% (w/w), less than about 70% (w/w), less than about 60% (w/w), less than about 50% (w/w), less than about 40% (w/w), less than about 30% (w/w), less than about 20% (w/w), less than about 15% (w/w), less than about 10% (w/w), less than about 5% (w/w), less than about 2% (w/w), less than about 1% (w/w), and less than about 0.1% (w/w) can be observed in the first permeate. These ranges of lipid content may also pertain to the miscella stream or crude cannabinoid extract (e.g., the product of extraction plus a single membrane extraction system).

Fluxes of the Permeate

Fluxes of the permeate through a membrane may range from about 0 L/h·m² to about 1000 L/h·m², from about 10 L/h·m² to about 750 L/h·m², from about 20 L/h·m² to about 500 L/h·m², from about 30 L/h·m² to about 450 L/h·m², from about 40 L/h·m² to about 400 L/h·m², from about 50 L/h·m² to about 350 L/h·m², from about 75 L/h·m² to about 300 L/h·m², from about 100 L/h·m² to about 250 L/h·m². Other suitable fluxes of the permeate through a membrane include ranges from about 0 L/h·m² to about 10 L/h·m², from about 10 L/h·m² to about 50 L/h·m², from about 50 L/h·m² to about 100 L/h·m², from about 100 L/h·m² to about 200 L/h·m², from about 200 L/h·m² to about 400 L/h·m², from about 400 L/h·m² to about 600 L/h·m², from about 600 L/h·m² to about 800 L/h·m², from about 800 L/h·m² to about 1000 L/h·m². Thus, the flux of a permeate through a membrane can be about 5 L/h·m², about 10 L/h·m², about 15 L/h·m², about 20 L/h·m², about 25 L/h·m², about 50 L/h·m², about 75 L/h·m², about 100 L/h·m², about 125 L/h·m², about 150 L/h·m², about 175 L/h·m², about 200 L/h·m², about 250 L/h·m², about 400 L/h·m², about 600 L/h·m², about 800 L/h·m², about 1000 L/h·m². These fluxes correspond to flow rates of 0-600 liters per hour, or higher depending on the scale of the process (e.g., membrane surface area).

Unexpectedly, it has been found that any pesticides and fungicides present in the miscella stream are retained with the lipids in the first retentate despite having molecular weights below the molecular weight cutoff of the first membrane. As noted above the amount of pesticides and/or fungicides applied to the Cannabis plants and the removal of any pesticides, e.g., by rinsing or washing, determines the concentration of any pesticides/fungicides extracted by the solvent with the cannabinoid and terpenes. One of the unexpected advantages of the system and methods described herein is that the Cannabis plant material does not require any drying, washing, rinsing, because the pesticides and fungicides are removed in the first retentate and do not contaminate the second retentate comprising the cannabinoids. Therefore, the concentration of pesticides or fungicides in a lipid rich retentate may range from about 0 ppm to about 1000 ppm, from about 0.0001 ppm to about 500 ppm, from about 0.001 ppm to about 400 ppm, from about 0.01 ppm to about 300 ppm, from about 0.1 ppm to about 200 ppm, from about 1 ppm to about 100 ppm, from about 5 ppm to about 50 ppm, or from about 10 ppm to about 25 ppm. The concentration ranges for pesticides or fungicides also include from about 0.1 ppm to about 10 ppm, from about 10 ppm to about 25 ppm, from about 25 ppm to about 50 ppm, from about 50 ppm to about 100 ppm, from about 100 ppm to about 200 ppm, from about 200 ppm to about 500 ppm, or from about 500 ppm to about 1000 ppm. Pesticide and fungicide concentrations within these ranges include about 0 ppm, about 0.0001 ppm, about 0.001 ppm, about 0.01 ppm, about 0.1 ppm, about 1 ppm, about 5 ppm, about 10 ppm, about 25 ppm, about 50 ppm, about 100 ppm, about 200 ppm, about 500 ppm, or about 1000 ppm.

The cannabinoid extract may comprise less than 0.5%, 0.1%, 0.01%, or 0.001% w/w pesticides, fungicides, fertilizers, and mixtures thereof. For example, the cannabinoid extract can comprise a concentration of pesticides or fungicides ranging from about 0 ppm to 10 ppm.

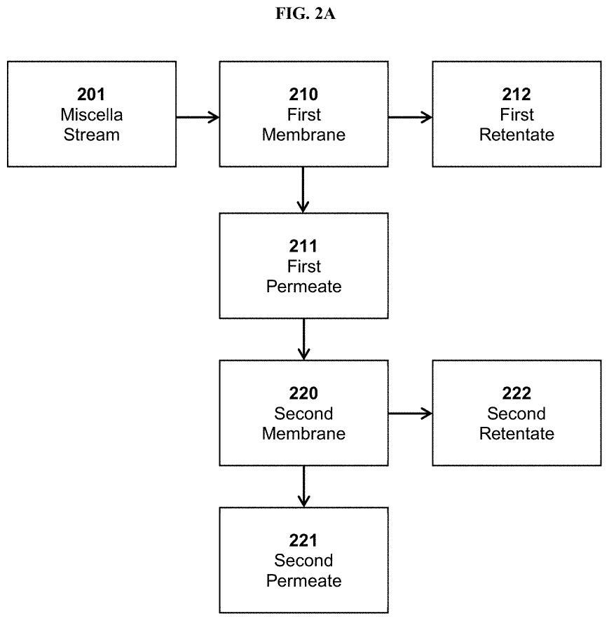

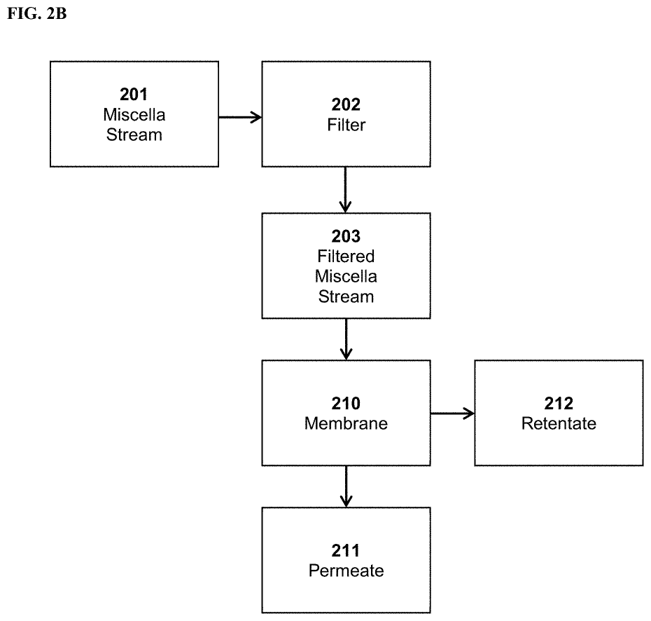

Making reference to FIG. 2A, the method includes subjecting miscella stream 201 as described herein to filtration and then a double membrane system 170 comprising a first membrane 210, e.g., with a molecular weight cutoff of about 500 Da and a second membrane 220, e.g., with a molecular weight cutoff of about 300 Da. The filtered miscella stream may be passed through a first membrane 210 to produce a first retentate 212 and first permeate 211. First retentate 212 includes lipids (e.g., fats, waxes, phospholipids, and fatty acids) and any pesticides/fungicides. The cannabinoids and terpenes flow through the first membrane with the first permeate 211 and the bulk of the solvent used to prepare miscella stream 201. The first permeate 211 is sent to a second membrane 220 to yield second retentate 222 in which the cannabinoids and terpenes are concentrated. Second permeate 221 includes the recovered solvent.

Another embodiment is depicted in FIG. 2B, the method includes subjecting miscella stream 201 as described herein to filtration 202 to produce a filtered miscella stream 203 which is then sent through a single membrane system 170 comprising a single membrane 210, e.g., with a molecular weight cutoff of about 200 Da. The retentate 212 includes the crude cannabinoid extraction comprising lipids (e.g., fats, waxes, phospholipids, and fatty acids), cannabinoids, and terpenes. The permeate 211 includes the recovered solvent, which may be filtered and recycled to the extraction tank.

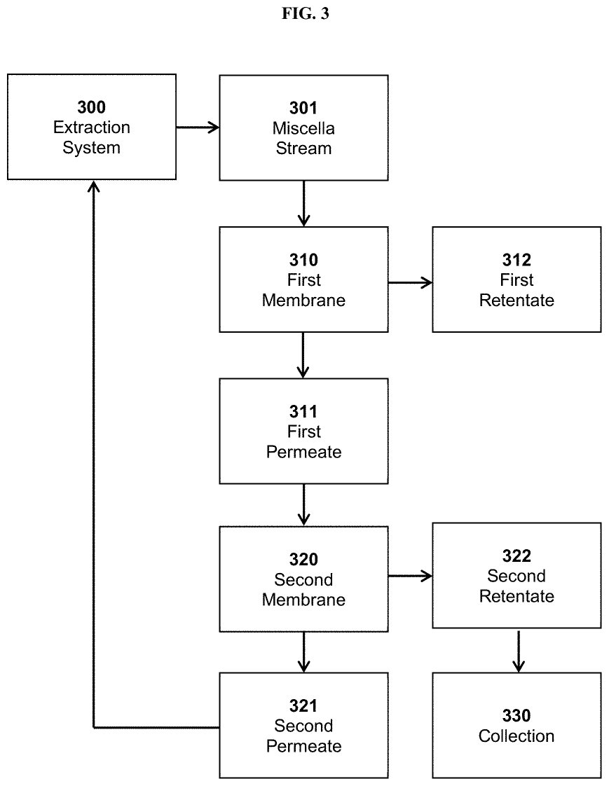

Another method is shown in the flow chart of FIG. 3. Extraction 300 can be performed by mixing pre-treated or untreated Cannabis plant into the solution with agitation (e.g., mixing) and subjecting it to sonication. The miscella stream 301 is then separated from the Cannabis plant material and allowed to flow through a drain for filtration. Following ultrasonic cavitation treatment, the miscella stream is subjected to a filtration step, and then the miscella stream 301 is passed through a first membrane 310. First retentate 312 includes lipids (e.g., fats, waxes, phospholipids, and fatty acids), any pesticides/fungicides. First permeate 311 includes the water used to prepare liquid 301, cannabinoids, and terpenes. First permeate 311 is then subjected to filtration through the second membrane 320 to yield second retentate 322, comprising the cannabinoids and terpenes that can be collected 330. Second permeate 321 includes the recovered solvent, which can be recycled back into extraction 300.

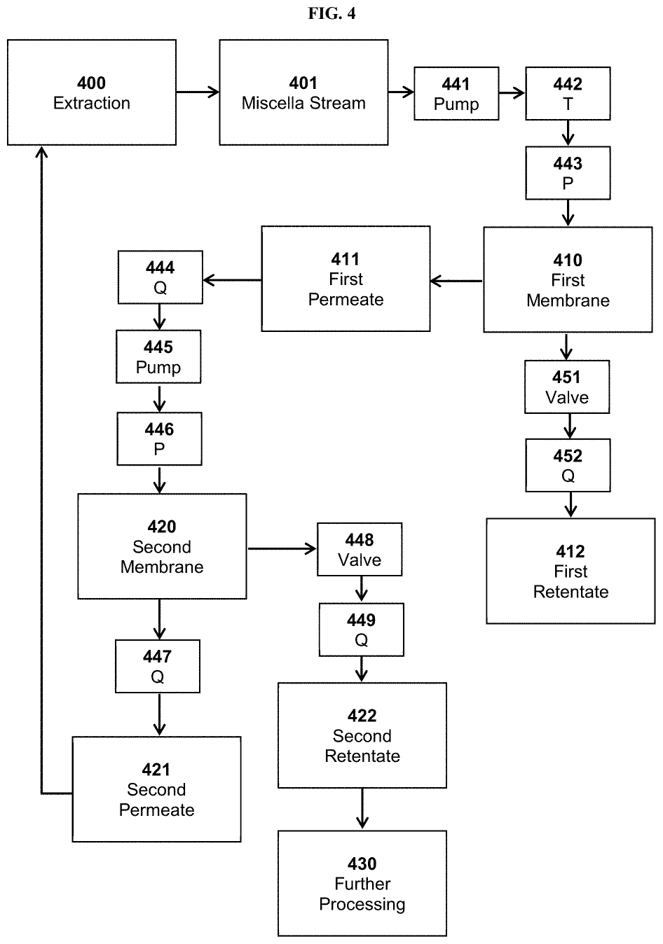

FIG. 4 shows another method of preparing cannabinoid extracts that includes a step of extraction 400, which can be performed using ultrasonic cavitation as described herein. The resulting miscella stream 401 is pumped using pump 441, the temperature of the liquid is measured using temperature gauge 442, and the pressure is measured using pressure gauge 443. Liquid 401 is then subjected to filtration step at the first membrane 410. First retentate 412, including captured lipids and any pesticides and fungicides, flows through back pressure valve 451, and flowmeter 452. Flowmeter 444 is used to measure the flow rate (Q) of first permeate 411, including the cannabinoids, terpenes, and solvent. Pump 445 pumps first permeate 411, and pressure gauge 446 measures the pressure of first permeate 411 before it is subjected to the second membrane 420. Flowmeter 447 measures the flow rate of second permeate 421, which includes the recovered solvent and is recycled into extraction 400. Second retentate 422 flows through back pressure valve 448, and the flow rate of second retentate 422 is measured using flowmeter 449. Second retentate 442 comprising the cannabinoids can be collected for further processing.

### THC Abatement

The cannabinoid composition produced by methods described herein may undergo tetrahydrocannabinol (THC) abatement, for example, by chromatography. In an embodiment, it is preferred that the substantially lipid free cannabinoid extract undergo THC abatement prior to decarboxylation. The inventor surprisingly found that the acid species of the cannabinoids are preferentially separated by chromatography. This leads to an unexpected improvement in separation of the cannabinoids, including tetrahydrocannabinol (THC), by chromatography.

### Cannabinol (CBD) Extraction, Decarboxylation, and Crystallization

The disclosure further provides for methods for decarboxylation and crystallization of cannabidiol acid (CBDA) to form cannabidiol (CBD):

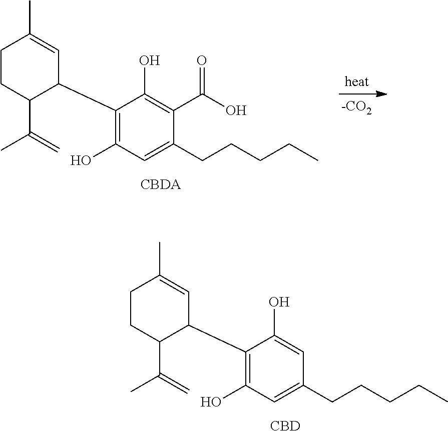

Other cannabinoids may also be decarboxylated using the methods described herein. For example, tetrahydrocannabinolic acid (THC) may be decarboxylated to form tetrahydrocannabinol (THC).

The decarboxylation (removal of —COOH species) from the cannabinoid acid to form a decarboxylated cannabinoid (e.g., the physiologically active form) and its subsequent crystallization may be performed using methods known in the art.

Crude Cannabis extract may have the lipids removed (e.g., winterized) prior to heat treatment to decarboxylate the cannabinoid acid to form decarboxylated cannabinoids (e.g., CBD or THC). It has been found that the lipid fraction comprising gums, resins, phospholipids, and oils, primarily traps the antifungal agents, pesticides, and fertilizers. By removing the lipids prior to decarboxylation and crystallization, these noxious agents can be excluded from the final product. The lipids may be removed by means of membranes, de-waxing, winterization methods known in the art, or a combination thereof.

A Cannabis extract may be heat treated to a sufficient temperature for about 1-60 minutes to decarboxylate a cannabinoid acid and form a cannabinoid. The cannabinoid may be collected, washed, or undergo a process for crystallization. The sufficient heat may be a temperature of about 100° C. to 160° C. The Cannabis extract may be heat treated for about 1-30 minutes. The Cannabis extract is substantially free of lipids. The Cannabis extract can comprise less than 1% lipids w/w. The cannabinoid may be substantially free of terpenes. The cannabinoid can comprise less than 1% terpenes w/w.

The cannabinoid crystals recovered after decarboxylation and crystallization may be substantially free of THC. For example, the cannabinoid crystal may comprise less than 0.3% THC w/w.

Further embodiments of the present invention will now be described with reference to the following examples. The examples contained herein are offered by way of illustration and not by any way of limitation.

### EXAMPLES

The methods described herein will now be described with reference to the following examples. The examples contained herein are offered by way of illustration and not by any way of limitation.

### Example 1

### Ultrasonic Treatment and Membrane Extraction of CBD from Hemp

### Sonification Rod (Horn)

A sonification horn operating at 20-100 kHz in an alcohol matrix demonstrated improvements to extraction yield and reduction in overall biomass solvent dwell time. Experimentation was performed in which a series of stirred extractions on 5 grams of Cherry wine Hemp (˜14.5% CBD) (aged about 8-10 months post-harvest) were conducted in 125 mL methanol and repeated in ethanol, using a frequency output of 35 kHz and an overall power output of 80 W for 5 minutes soak and 30 second sonication. The samples were filtered through a 5 μM filter paper in a Buchner funnel. Samples were taken of each collected filtrate (miscella stream).

TABLE 1
ULTRASOUND EXTRACTION DATA

| Power Output (W) | 80 |
|---|---|
| Frequency (kHz) | 35 |
| Biomass Amount (g) | 5 |
| Hemp Strain | Cherry Wine |
| Assay CBD-A (%) | 13.52 |
| Solvent Volume (mL) | 125 |
| Soak Time (min.) | 5 |
| Ultrasonic Treatment (seconds) | 30 |
| Solvent/Ultrasonic Treatment | CBD-A Extraction Yield |
| Ethanol- No Ultrasonic Treatment (mg/g) | 2.94 |
| Ethanol + Ultrasonic Treatment (mg/g) | 4.80 |
| Methanol- No Ultrasonic Treatment (mg/g) | 3.46 |
| Methanol + Ultrasonic Treatment (mg/g) | 5.14 |

CBD-A extraction yield in the filtrate was determined by High Pressure Liquid Chromatography (HPLC) analysis performed on each sample to determine CBD-A yield in the collected miscella.

### Two-Stage Membrane Filtration

The miscella stream was then pumped through a series of filters to remove residual post-extraction particles. The filtered miscella stream was then pumped through a series of suitable semi-permeable membranes. The first membrane in this series is selected by pore size (500 Daltons) to reject any materials larger than the selected membrane pore such as fats, gums, waxes, chlorophyll and chloroplasts that were extracted in the screw conveyor. The cannabinoids and solvent permeate this first stage, and then be separated by a second-stage membrane in the same described fashion. The second retentate can be sent for further post-processing and the permeated solvent would then be thermostatically prepared at the selected extraction temperature and return to the inclined screw conveyor extractor and distributed throughout the process via spray nozzles along the top of the extraction conveyor.

A series of trials were performed on flat sheet samples supported on a Sterlitech Inc. CF016 flat sheet membrane device. Two 5-gallon extractions were performed on hemp biomass using ethanol and methanol for each extraction. The biomass material was filtered and the miscella was deposited and sealed in a steel pressure vessel. The solvent output was connected to the flat sheet membrane housing and an adjustable needle valve was connected to the flat sheet membrane reject port to control back-pressure. The vessel was charged with nitrogen at various pressures causing the filtered miscella to apply pressure to the membrane input. Permeate solvent was collected in a graduated cylinder, with collection rates measured hourly.

### HPLC Data

The ultrasonic treatment surprisingly leads to a 61.3% increase in cannabinoid yield in ethanol and 67.3% increase in cannabinoid yield in methanol, as compared to no ultrasonic treatment. Further, it was unexpected that methanol would perform better than ethanol in cannabinoid extraction. It has been surprisingly discovered that despite the age of the hemp used in the experiment (about 8-10 months post-harvest) the solvent and ultrasonic conditions used were successful in removing the trichomes but not disrupting the Cannabis plant matter. While not wishing to be bound by any specific theory, it is believed that the age of the hemp lead to polymerization of the lipids in the trichomes making the cannabinoids resistant to extraction. It was unexpected when a relatively low ultrasonic setting at a short time period lead to nearly complete removal of the trichomes from the plant material. This leads to an advantage of not releasing organelles, nucleic acids, fibers, or lignin which tend to foul membranes in the downstream extraction steps. This greatly reduces the amount of biomass processed after the first step, saving time and costs for CBD extraction from Cannabis.

### EXAMPLE 2

### Ultrasonic Cavitation of Hemp at Different Temperatures

### Sonification Rod (Horn)

The inventor performed a series of extractions at several temperatures (20° C., 0° C., and −60° C.) using a sonification horn operating at 20-100 kHz in an alcohol solvent with temperature recording to better determine ultrasonic extraction efficacy as a function of temperature. Experimentations were performed in which a series of stirred extractions on 0.1 grams of Auto Tsunami Hemp (4.3% available CBD) (aged about 15 days post-harvest) in 30 g methanol were sonicated using a frequency output of 35 kHz and an overall power output of 100 W for 6 10 second pulses for a total contact time of 3 minutes. Each sample was filtered using a 0.22 um syringe filter and injected into the HPLC for concentration analysis.

TABLE 2
Concentration measurements

| | |
|---|---|
| Temperature | % of Available CBD released |

| | | |
|---|---|---|
| 20° | C. | 96% |
| 0° | C. | 37% |
| −60° | C. | 41% |

HPLC measurement showed increased CBDA solubility at 20° C. versus 0° C. and −60° C. This suggests that methods run at lower temperature risk lower extraction efficiency because of the poor solubility of CBDA (the primary species of CBD in unprocessed hemp) at lower temperatures. This is unexpected because it is contrary to standard methods in the art which rely on below 0° C. temperatures to winterize lipids for removal.

The inventor surprisingly discovered that, contrary to the current position of the art, ultrasonic extraction performance deteriorates as temperature is reduced, dissimilar CBD, CBDA solubility increased as a function of temperature, and delta-9 THC measured after ultrasonic application was nearly identical at all temperatures in defined range.

The claimed method showed a 55% better result at 20° C. than 0° C. and a 59% improvement over −60° C. This allows the claimed system and method to be commercialized in a smaller footprint than existing systems because it does not require extensive chillers. This also greatly reduces operating costs as the system need only be maintained between 10° C. and 30° C., instead of at temperature around −60° C.

### EXAMPLE 3

### Ultrasonic Cavitation of Hemp

### Sonification Rod (Horn)

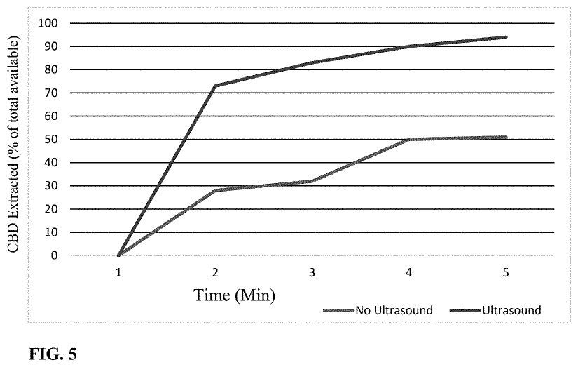

The inventor performed a series of extractions at several temperatures (5° C.) using a sonification horn operating at 20-100 kHz in an alcohol solvent with temperature recording to compare extraction via ultrasonic extraction versus soaking in a solvent. Experimentations were performed in which a series of stirred extractions on 5 grams of Auto Tsunami Hemp (4.3% available CBD) (aged about 15 post-harvest) in 100 g methanol were sonicated using a frequency output of 35 kHz and an overall power output of 80 W for 30 second pulses for a total contact time of 5 minutes. Each sample was filtered using a 0.22 μm syringe filter and injected into the HPLC for concentration analysis. See FIG. 5.

HPLC measurement showed about 95% CBD extraction using ultrasound by 4 minutes, versus about 51% without ultrasound. This suggests that the ultrasound allows for rapid release of the CBD into the alcohol solvent in a relatively short contact time. This is unexpected because it is contrary to standard methods in the art which rely on long contact time, sometimes hours of contact time to extract CBD, at much lower temperatures, e.g., −40° C.

### Definitions

Unless defined otherwise, all technical and scientific terms used herein have the same meaning as those commonly understood by one of ordinary skill in the art.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the steps can be carried out in any order without departing from the principles of this disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

As used herein, “trichome” generally refers to a fine outgrowth or appendage on plants and certain protists. They are of diverse structure and function. Examples are hairs, glandular hairs, scales, and papillae. In reference to Cannabis, there are three types of trichomes that develop in the Cannabis plant. These include:

“Bulbous trichomes,” as used herein, refers broadly to trichomes that appear as small pointed structures on the surface of the plant. They are the smallest trichomes and are responsible for secreting resins.

“Capitate-sessile,” as used herein, refers broadly to a type of trichomes is bigger compared to bulbous trichomes and tend to develop before the plant starts flowering. They are flattened and contain cannabinoids.

“Capitate stalked trichomes,” as used herein, refers broadly to the largest of the three types of trichomes and form during flowering. They are largely involved in the synthesis of cannabinoids and terpenoid synthesis.

“Cannabinoid acid,” as used herein, refers broadly to the chemical precursor of an active cannabinoid. Generally, the cannabinoids produce in situ by Cannabis are present in an acid form. These can be heated to decarboxylate, and active, the cannabinoid. Common cannabinoid acids include but are not limited to cannabidiol acid (CBDA) and tetrahydrocannabinol acid (THCA), e.g., Δ⁹ and Δ⁸ tetrahydrocannabinol acid.

“Cannabidiol acetate,” as used herein, refers broadly to the chemical precursor of cannabidiol. This is also referred to as cannabidiol acid, or CBDA in the art:

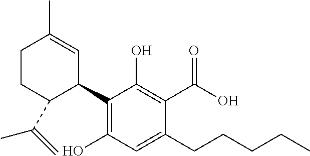

CBDA is heated to decarboxylate and form cannabidiol (CBD).

“Cannabis plant material,” “Cannabis,” and “Cannabis material,” as used herein, refers broadly to any Cannabis plant or part thereof, this includes but is not limited to, flowers, stems, nodes, leaves, pistils, colas, calyxs, trichomes, seed, stalk, buds (including dormant buds, axillary buds, and terminal buds), petiole, rachis, bract, and roots. Cannabis plant material also refers broadly to hemp that includes but is not limited to Cannabis plants with less than 0.3% THC content. Hemp and industrial hemp can be used interchangeably as both refer to Cannabis plants with less than 0.3% THC content.

“Cannabis,” as used herein, refers broadly to all plants of the genus Cannabis and/or the family cannabaceae, including but not limited to all plants of the species Cannabis sativa, Cannabis indica, and Cannabis ruderalis. Hybrids, clones, cultivars, and varieties are also included. Cannabis also broadly includes hemp.

“Crude cannabinoid extract,” as used herein, refers broadly to a cannabinoid solution comprising cannabinoids, terpenes, and lipids.

“Lipids,” as used herein, refers broadly to waxes, gums, fats, and mixtures thereof. The term also encompasses mono-, di- and triacylglycerols, phospholipids, free fatty acids, fatty alcohols, cholesterol, cholesterol esters, and the like. The term “fatty acid” refers to a carboxylic acid with a long aliphatic tail of at least 8, at least 10, at least 12, at least 16, at least 18 or at least 22 carbon atoms in length, either saturated or unsaturated. Examples of fatty acids include linear fatty acids of C₆-C₂₄ such as caproic acid, caprylic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, erucic acid, and mixtures thereof.

“Phospholipid” as used herein refers to a glycerol phosphate with an organic headgroup such as choline, serine, ethanolamine or inositol and zero, one or two (typically one or two) fatty acids esterified to the glycerol backbone. Phospholipids include, but are not limited to, phosphatidylserine, phosphatidylglycerol, phosphatidylcholine, phosphatidylethanolamine, phosphatidylserine, phosphatidylinositol, and diphosphatidylglycerol as well as corresponding lysophospholipids.

“Miscella stream” and “miscella,” as used herein, refers broadly to a solvent comprising mixture of lipids and organic compounds (e.g., cannabinoids).

“Molecular weight cutoff,” as used herein, refers broadly to the minimum molecular weight of a solute that is 90% retained by a membrane. See, e.g., K. J. Kim et al., Journal of Membrane Science 87: 35-46 (1994) using dextran and a transmembrane pressure of 50 kPa.

“Ultrasonics” or “ultrasonic waves,” as used herein, refers broadly to sound waves (mechanical waves) with high frequency about between 15 kHz to 40 kHz (e.g., about 20 kHz) and low amplitude of about between 0.0001-0.025 mm.

“Winterization,” as used herein, refers broadly to any means by which lipids are removed from a cannabinoid extract.

Although the subject matter disclosed herein has been described in some detail by way of illustration and example for purposes of clarity of understanding, it should be understood that certain changes and modifications can be practiced within the scope of the appended claims. Modifications of the above-described methods would be understood in view of the foregoing disclosure or made apparent with routine practice or implementation of the described methods to persons of skill in extraction chemistry; extraction processing, mechanical engineering, and/or related fields are intended to be within the scope of the following claims.

All publications (e.g., non-patent literature), patents, patent application publications, and patent applications mentioned in this specification are indicative of the level of skill of those skilled in the art to which this disclosure pertains. All such publications (e.g., non-patent literature), patents, patent application publications, and patent applications are herein incorporated by reference to the same extent as if each individual publication, patent, patent application publication, or patent application was specifically and individually indicated to be incorporated by reference.

While the foregoing methods have been described in connection with this disclosure, it is not to be limited thereby but is to be limited solely by the scope of the claims which follow.

1-195. (canceled)

196. A method for processing Cannabis plant material comprising
combining Cannabis plant material and at least one solvent in an extraction tank to produce a miscella stream,
wherein the extraction tank comprises a screw conveyor located inside the extraction tank, the screw conveyor capable of moving the Cannabis plant material from the first end to the second end,
applying ultrasonic cavitation with an ultrasonic cavitation device to the mixture of the Cannabis plant material and at least one solvent in the extraction tank to produce a miscella stream;
filtering the miscella stream through at least one filter;
filtering the filtered miscella stream through at least one membrane to produce a cannabinoid extract.

197. The method of claim 196, wherein the sonication device is horn, probe, cup, or rod shaped.

198. The method of claim 196, wherein the sonication device produces an ultrasound frequency between 1 kHz to 100 kHz.

199. The method of claim 196, wherein the ultrasonic cavitation is applied for 1-180 seconds.

200. The method of claim 196, wherein the method further comprising moving the Cannabis plant material to an output port located at the second end.

201. The method of claim 1, wherein the screw conveyor is a varied geometry screw conveyor.

202. The method of claim 1, wherein the screw conveyor has a central axis, the central axis making an incline angle θ of about 1° to about 90° C. relative to a horizontal plane intersecting the central axis.

203. The method of claim 1, wherein the solvent is an alcohol.

204. The method of claim 203, wherein the alcohol is methanol, ethanol, n-propanol, 1-propanol, nbutanol, sec-butanol, t-butanol, 1-pentanol, anyl alcohol, 1-hexanol, 2-hexanol, 3-hexanol, 1-heptanol, 2-heptanol, 3-heptanol, 4-heptanol, 1-octanol, 2-octanol, 1-nonanol, 1-decanol, or a mixture thereof.

205. The method of claim 204, wherein the alcohol is methanol, ethanol, or a mixture thereof.

206. The method of claim 205, wherein the mixture comprising Cannabis plant material and at least one solvent has a temperature between −25° C. and 25° C.

207. The method of claim 206, wherein the membrane is an organic solvent resistant membrane.

208. The method of claim 207, wherein the membrane is a silicone membrane, polymeric organophilic membrane, polyimide membrane, optionally modified polyimide membrane, polyflouride membrane, polyether-ether ketone membrane, methyl-ethyl ketone (MEK) membrane, polyethylene membrane, polypropylene membrane, cellulose acetate membrane, polystyrene membrane, polytetrafluoroethylene membrane, polyimide membrane, or polysilane membrane.

209. The method of claim 207, wherein the membrane has a molecular weight cutoff of between about 100 Daltons and 1,100 Daltons.

210. The method of claim 207, wherein the filter has a pore size of between about 0.01 and 10 μm.

211. The method of claim 196, wherein the solvent is removed from the cannabinoid extract, including retentates, by means for solvent removal.

212. The method of claim 196, wherein the Cannabis plant material is washed, dried, comminuted, or a combination thereof.

213. The method of claim 196, wherein the method is a continuous method.

214. The method of claim 196, wherein the method is practiced on a mobile extraction platform.

215. The method of claim 196, wherein the method further comprises heating the Cannabis extract for about 1-60 minutes a sufficient temperature to decarboxylate a cannabinoid acid to form a cannabinoid; and collecting the cannabinoid.

216. A system for processing Cannabis plant material comprising an extraction tank coupled to a membrane system, wherein the system comprises an ultrasonic cavitation device configured to apply ultrasonic cavitation to a mixture of Cannabis plant material and at least one solvent in the extraction tank comprising a screw conveyor, wherein the extraction tank is in fluid communication with a filter, membrane system, and solvent extraction means.
